(19) United States
(12) Patent Application Publication
(10) Pub. No.: US 2015/0165184 A1
(43) Pub. Date: Jun. 18, 2015

(54) System and Method for Concerted Operation of Biopsies and Other Medical Tasks

(71) Applicants: William Schulenberg, Chicago, IL (US); Preeti Sukerkar, Palo Alto, CA (US); Dhwanil Damania, San Jose, CA (US); John Saran, Chicago, IL (US); Deniz Alpay, Chicago, IL (US); Richard Ahn, Dallas, TX (US)

(72) Inventors: William Schulenberg, Chicago, IL (US); Preeti Sukerkar, Palo Alto, CA (US); Dhwanil Damania, San Jose, CA (US); John Saran, Chicago, IL (US); Deniz Alpay, Chicago, IL (US); Richard Ahn, Dallas, TX (US)

(21) Appl. No.: 14/568,102

(22) Filed: Dec. 11, 2014

Related U.S. Application Data
(60) Provisional application No. 61/915,484, filed on Dec. 12, 2013.

(51) Int. Cl. A61M 39/02; A61B 17/34; A61M 5/32; A61B 10/04

(52) U.S. Cl. CPC A61M 39/0247; A61B 10/04; A61B 17/3423; A61B 17/3439; A61M 5/3287; A61B 2017/3447; A61M 2039/0205; A61M 2039/0202; A61M 2039/0264; A61B 2010/045

(57) ABSTRACT

The present invention relates to a system and method for utilizing a primary component and an auxiliary component in concert upon locations in close proximity to one another. Examples of benefits from this invention include medical applications such as biopsy procedures. Other benefits include minimization of cancerous cell seeding by using embolic material to block the spread of cancer cells from biopsied locations. Other benefits include detection of tissues, or masses such as cancerous tumors, through the use of a contrast agent. Additional benefits include prevention of bleeding or other migration of biological material from sites that have been medically operated on. Other benefits include blocking direct contact between a biopsy stylet which has taken a biopsy sample, and the walls of a path within the patient through which said biopsy stylet is withdrawn afterwards; this can minimize risks of cancerous debris being spread along said path.

This application claims priority from, relies upon, and relates to Provisional Application No. 61/915,484, filed on Dec. 12, 2013.

### BACKGROUND OF THE INVENTION

Currently, tasks such as percutaneous medical operations involve both risks and inconveniences. Some of these risks include the risk of cancerous cell seeding, which can occur when a biopsy procedure creates loose cancer cells, and which can result in these cells spreading to undesired locations in a patient's body. This mechanism of cell seeding is linked in medical literature to the metastasis of cancerous cells. The problem of cell seeding is particularly pronounced where the type of cancer being biopsied has a high incidence of seeding, such as cancers of the breast, liver or kidney.

Other risks and inconveniences of tasks such as percutaneous medical operations include the incidence of bleeding due to the operation, as well as risks that fluids or other biological material will spread in undesired ways due to said medical operations.

Current techniques for minimizing the risk of cell seeding, the risk of bleeding, and the risks of spreading biological material due to medical operations, may involve injection of embolic agents or other material. However, current techniques may include the use of two separate instruments, and attempting to use two separate instruments in concert with one another can create logistical and operational issues. Due to the limitations of these instruments or due to the difficulties of operating the two instruments in concert, such techniques do not always adequately reduce cell seeding, bleeding, or other complications.

Current techniques for conducting biopsies of potentially cancerous biological matter often comprise a biopsy stylet being inserted in a manner that creates a needle tract, and withdrawn through this tract in the opposite direction after obtaining a biopsy sample. However, this poses the risk that cancerous debris or other hazardous matter will fall off of the biopsy stylet as said biopsy stylet is being withdrawn.

Currently, tasks such as medical imaging involve inefficiencies and inaccuracies, some of which are due to the logistical and operational difficulties which are inherent in the attempts to coordinate multiple medical instruments. This is particularly the case when the medical imaging tasks in question involve percutaneous and/or in situ operations, as is sometimes the case, for instance, during surgery or when performing a biopsy. These difficulties may also arise when the medical imaging tasks in question require the injection of a contrast agent in conjunction with other operations or tasks. In such cases, the use of multiple instruments can limit the efficiency and/or accuracy of such medical imaging.

In general, many primary tasks such as medical operations can be enhanced through the performance of auxiliary tasks in close proximity to the site of said primary tasks. These auxiliary tasks may include injecting, or otherwise placing, substantive materials or agents in proximity to the mass being biopsied. By way of example, the use of embolic agents or other materials can minimize the risk of cell seeding, a mechanism linked in medical literature to the metastasis of cancerous cells. This is especially helpful when conducting biopsies of cancerous cells with a high incidence of seeding, such as cancers of the breast, liver and kidney. Additionally, the use of embolic agents or other materials can provide a buffer through which the biopsy is taken, which can reduce bleeding or provide similar benefits. Additionally, the use of contrast agents can enhance the ability to detect the tumor or other mass.

What's more, many primary tasks such as medical operations can be enhanced through the use of an appropriately designed primary component. One example could be a cannula or other type of needle which is adapted to accommodate said biopsy stylet within it. Such a cannula or needle could block direct contact between the biopsy stylet and the sides of the needle-created tract or cavity, and this could help protect against the risk that mechanically dragging said biopsy stylet out of a patient, through the needle tract or cavity, would lead to cancerous debris being spread.

### SUMMARY OF INVENTION

The present invention comprises systems and methods which are designed to allow a primary component, such as a biopsy needle or other medical instrument, to operate in combination with, in concert with, and in close proximity to an auxiliary component, such as a channel adapted for the transport of substances or other material. This invention enables the performance of primary tasks through the use of the primary component, while also enabling the performance of auxiliary tasks in close proximity to the area being acted upon through the primary task.

Examples of applications of this invention include using the primary component to conduct biopsies or other medical operations while using the auxiliary component to insert and/or remove a substance or material. Said substance or material might comprise an embolic material whose purpose is to block the spread of cancer cells from a tumor being biopsied, or an embolic material whose purpose is to reduce bleeding or other unwanted migration of biological material, or a contrast agent whose purpose is to facilitate detection, localization, or identification of biological tissues or masses such as cancerous tumors.

One advantage of the present invention is to limit difficulties involved in the operation of multiple instruments by allowing a user to use one system, which comprises a primary component and a secondary component, to perform primary and secondary tasks in concert with one another and in close proximity to one another.

An additional advantage of the present invention is to provide improved systems and methods for biopsies of such materials as fluids, or tissues such as cancerous tumors or other masses.

Another advantage of the present invention is to provide improved systems and methods for percutaneous needle biopsies of other unknown masses or collection of fluid in the human body.

A further advantage of the present invention is to reduce the contact of the system in general with a cancerous tumor or unknown mass cells, due to the system comprising a biopsy device with increased mobility and with a smaller cross-section than the system as a whole.

Another advantage of the present invention is to reduce the contact of medical instruments such as biopsy devices with the tissue of a patient as said medical instrument is inserted or withdrawn. This can be particularly helpful in, for example, preventing a biopsy stylet from contacting walls of a needle tract or other cavity as said biopsy stylet is removed following its taking of a biopsy sample. Such an advantage can minimize the spread of biopsied material such as cancer cells, which might otherwise be spread as said biopsy stylet is taken out of the patient.

Similarly, an advantage of the present invention is to provide for the reduction of bleeding and the sealant of a biopsy hole or cavity through the introduction of embolic agent materials. This can be particularly advantageous in that it can limit the mobility of cancerous debris or other potentially hazardous matter.

Furthermore, an advantage of the present invention is to allow improved visualization of the cancerous tumor or unknown mass via imaging through the introduction of contrast agents.

Still a further advantage of the present invention is to provide a method that decreases long-term economic costs to patients, providers, and other stakeholders by lowering the incidence of manmade metastasis of cancerous tumor cells.

Moreover, an advantage of the present invention is to provide a method that increases positive clinical outcomes by lowering the incidence of man-made metastasis of cancerous tumor cells.

Various specifications, features and advantages of the present invention are and can become more apparent upon review of this disclosure including the asserted claims with specific references to the various drawings. The advantages are always desired, but not necessary to practice the present invention.

### DETAILED DESCRIPTION OF THE INVENTION

The present invention comprises a system, said system comprising a primary component and an auxiliary component which are adapted to work in concert and which are adapted to perform primary and auxiliary tasks upon physical locations which are in close proximity to one another, said primary component comprising a distal end and a proximal end, said auxiliary component comprising a distal end and a proximal end, said distal end of said primary component and said distal end of said auxiliary component being adapted to be capable of being positioned in close proximity to one another.

In some embodiments, said primary component comprises an instrument or path.

In some embodiments, said auxiliary component comprises an instrument or path.

In some embodiments, said instrument or path comprises a hypodermic needle, a biopsy apparatus, or a fluid delivery tube.

In some embodiments, said primary component comprises a first access port and a first cannula which interface with one another, and said auxiliary component comprises a second access port and a second cannula which interface with one another.

In some embodiments, each of the two said access ports comprises a distal end which is adapted to interface with a cannula, and a proximal end which is adapted to allow external devices or materials to be directed through said access port into said cannula.

In some embodiments, said first cannula comprises a proximal end and a distal end, said second cannula comprises a proximal end and a distal end, said distal ends of each cannula are adapted to be capable of being positioned in close proximity to one another, said proximal end of the first cannula is adapted to interface with the distal end of the first access port, and said proximal end of the second cannula is adapted to interface with the distal end of the second access port.

In some embodiments, the first access port is adapted to receive a medical instrument, said medical instrument comprising the first cannula.

In some embodiments, said medical instrument comprises a hypodermic needle or biopsy apparatus.

In some embodiments, said medical instrument is adapted to accommodate a second medical instrument.

In some embodiments, said second medical instrument comprises a hypodermic needle or biopsy apparatus.

In some embodiments, said first cannula and said second cannula are integrated into a combined cannula needle.

In some embodiments, one or both cannulas comprise rigid material.

In some embodiments, one or both cannulas comprise elastic material.

In some embodiments, one of the two cannulas comprises rigid material, and the other of the two cannulas comprises elastic material.

In some embodiments, a separation barrier separates one cannula from the other cannula.

In some embodiments, said separation barrier is planar or roughly planar.

In some embodiments, said separation barrier is a cylindrical shape or other enclosed shape.

In some embodiments, said separation barrier comprises the first cannula.

In some embodiments, said first cannula comprises an inner cannula and said second cannula comprises an outer cannula.

In some embodiments, said inner cannula will comprise rigid material, and said outer cannula will comprise elastic material.

In some embodiments, said outer cannula will comprise an elastic membrane which is adapted to surround said inner cannula.

In some embodiments, said outer cannula will be adapted to change shape in response to internal pressure, such as pressure created by viscous materials or other substantive materials which are capable of being directed in between said membrane and said inner cannula.

In some embodiments, the first and second access ports are physically attached in a single-piece unit.

In some embodiments, the first and second access ports are separate components.

In some embodiments, the first and second access ports are adapted to be capable of being physically secured to each other.

In some embodiments, one or both access ports will comprise an attachment mechanism which is adapted to secure one access port to the other access port.

In some embodiments, one access port will be adapted to fit completely or partially into a space within the other access port.

In some embodiments, one access port comprises an inner body and the other access port comprises an outer body, said inner body being adapted to fit completely or partially into a space within the outer body.

In some embodiments, the second cannula is adapted to fit between said inner body and said outer body, and is also adapted to extend out of the distal opening of the second access port.

In some embodiments, the second cannula comprises additional adaptations which enable it to better fit into the space the inner body and the outer body.

In some embodiments, said additional adaptations comprise an opening or openings, each said opening being flush with other components of the system.

In some embodiments, one or both access ports are physically attached or secured to the cannulas with which they are adapted to interface.

In some embodiments, one or both access ports are separate from the cannulas with which they are adapted to interface, but one or both of said access ports are capable of being physically attached and/or secured to said cannulas.

In some embodiments, the proximal end or ends of one or both of said access ports have adaptations which make them capable of being secured to other objects or devices.

In some embodiments, one or both of said adaptations will comprise a screw shape.

In some embodiments, one or both access ports are adapted to comprise or interface with one or more sub-ports which are adapted to be capable of sub-functions.

In some embodiments, said sub-functions include acting as a filter or interfacing with a filter.

In some embodiments, said sub-ports comprise components which make them capable of being secured to other objects or devices.

In some embodiments, said components of said sub-ports comprise a screw shape.

In some embodiments, the second access port comprises a proximal end which is designed to interface with a syringe, said syringe being capable of injecting substantive materials.

In some embodiments, the first and second access ports are physically attached in a single-piece unit.

In some embodiments, said inner body and/or said outer body comprise a tapered shape.

Each of the features in the above embodiments are combinable and/or interchangeable with features from other embodiments. The ways in which the above embodiments may be combined and/or interchanged will be apparent to those of ordinary skill in the art in view of the teachings herein.

Methods of utilizing the system comprise steps of operating the primary component and steps of operating the auxiliary component.

In some methods, these steps are performed concurrently.

In some methods, these steps are performed consecutively.

Operating the primary component may comprise any method of navigating all or part of said primary component to a first location.

Operating the primary component may comprise any method of navigating the distal end of said primary component to a first location.

Operating the auxiliary component may comprise any method of navigating all or part of said auxiliary component to a second location.

Operating the auxiliary component may comprise any method of navigating the distal end of said auxiliary component to a second location.

Operating the primary component may comprise any method of conducting a biopsy or other medical operation.

Operating the auxiliary component may comprise any method of inserting a type of substantive material.

In some methods, operating the primary component comprises the step of navigating the distal end of said primary component to a first desired location, and operating the auxiliary component comprises the step of navigating the distal end of said auxiliary component to a second desired location which is in close proximity to said first desired location.

In some methods, the method comprises an additional step of utilizing the auxiliary component to insert substantive material, and the additional step of utilizing the primary component to conduct a biopsy or other medical operation.

In some methods, the method comprises an additional step of utilizing the auxiliary component to deliver substantive material to the area in which said biopsy or other medical operation was conducted.

In some methods, the method comprises an additional step of utilizing the auxiliary component to remove substantive material.

In some methods, said substantive material comprises embolic material, contrast agent, or sterile rinse.

In some methods, said method of navigating all or part of said primary component to a first location comprises a step of navigating said primary component through a cannula-style barrier.

In some methods, the method comprises an additional step of withdrawing said primary component through said cannula-style barrier.

### BRIEF DESCRIPTION OF THE DRAWINGS

FIGS. 1, 2 and 3 show a representative embodiment of the system. In said embodiment, a first access port and a second access port are attached in a single-piece unit. Also in said embodiment, each of the two said access ports comprises a proximal end and a distal end. Also in said embodiment, a first cannula and a second cannula are integrated into a combined cannula needle.

FIG. 1 shows said single-piece unit 100 which comprises a first access port, said first access port comprising a proximal end 105 which is adapted to allow external devices or materials to be directed through said access port. Said single-piece unit also comprises a second access port, said second access port comprising a proximal end 115 which is adapted to allow external devices or materials to be directed through said access port. Said single-piece unit also comprises an opening 110 which is adapted to interface with a combined cannula needle 120.

FIG. 2 shows a cutaway view of said single-piece unit 100 which comprises a first access port, said first access port comprising a proximal end 105 and a distal end 106. Said single-piece unit also comprises a second access port, said second access port comprising a proximal end 115 and a distal end 116. Said single-piece unit also comprises an opening 110 which is adapted to interface with a combined cannula needle 120. Said combined cannula needle 120 comprises a first cannula 200 and a second cannula 210. In this embodiment, the distal end 106 of the first access port is adapted to interface with the first cannula 200, and the distal end 116 of the second access port is adapted to interface with the second cannula 210. Also in this embodiment, the proximal end 105 of the first access port is adapted to allow external devices or materials to be directed through said first access port into said first cannula 200, and the proximal end 115 of the second access port is adapted to allow external devices or materials to be directed through said second access port into said second cannula 210.

FIG. 3 shows a cut-away view of said combined cannula needle 120 in addition to showing two magnified areas of same. A separation barrier 305 separates the first cannula 200 from the second cannula 210.

FIGS. 4, 5, 6, 7, 8, 9, 10, 11, 12, and 13 show another representative embodiment of the system. In said embodiment, the first access port comprises an inner body, the second access port comprises an outer body, and a catheter sheath comprises the second cannula. In this embodiment, these components are assembled into a system which is adapted to receive a medical instrument, which will comprise the first cannula once it is received. Such medical instrument might be a hypodermic needle; otherwise, another medical instrument can be substituted into this assembly and that medical instrument will comprise the first cannula.

FIG. 4 shows an inner body 2010, an outer body 2020, a catheter sheath 2030, and a hypodermic needle or other medical instrument which is represented by two dashed lines 2040. The catheter sheath 2030 is located in the interstitial space between the inner body 2010 and the outer body 2020. The hypodermic needle or other medical instrument, represented by the two dashed lines 2040, is inserted into and received by the inner body 2010. Said medical instrument 2040 will comprise the first cannula.

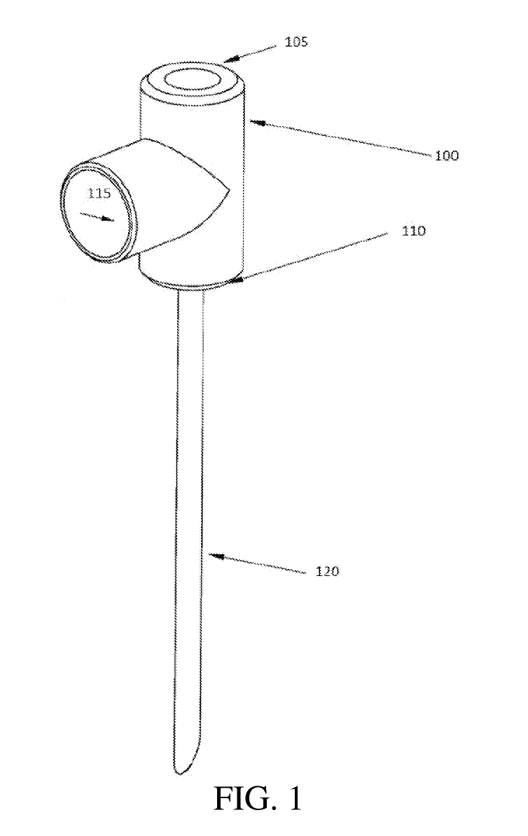

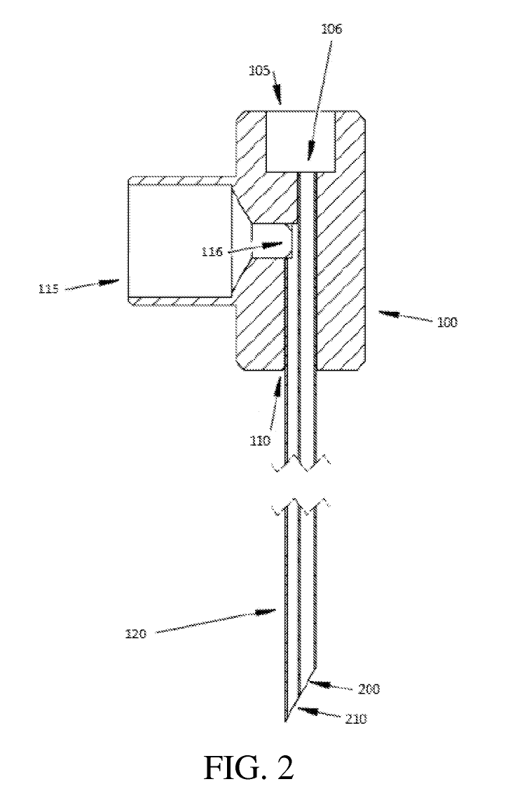

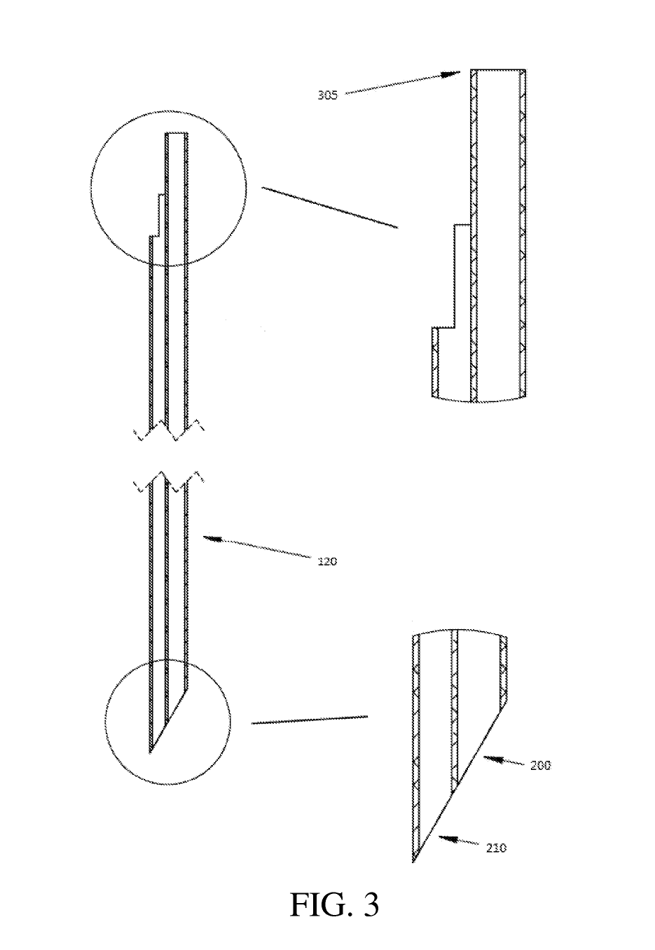

The inner body 2010 and outer body 2020 comprise adaptations to be physically secured to one another, and an attachment mechanism is comprised of these adaptations; said attachment mechanism comprises angled pegs 2021 on the outer body 2020, and also comprises an opposite-angled band 2011 on the inner body 2010.

The inner body 2010 is adapted to receive a medical instrument such as a hypodermic needle, said medical instrument being represented in this diagram by two dashed lines 2040. Said medical instrument 2040 will comprise the first cannula. Once said medical instrument is in place, said inner body 2010 is adapted to allow external devices or materials to be directed into the first cannula.

The catheter sheath 2030 is located in the interstitial space between the inner body 2010 and the outer body 2020. Said catheter sheath comprises the second cannula.

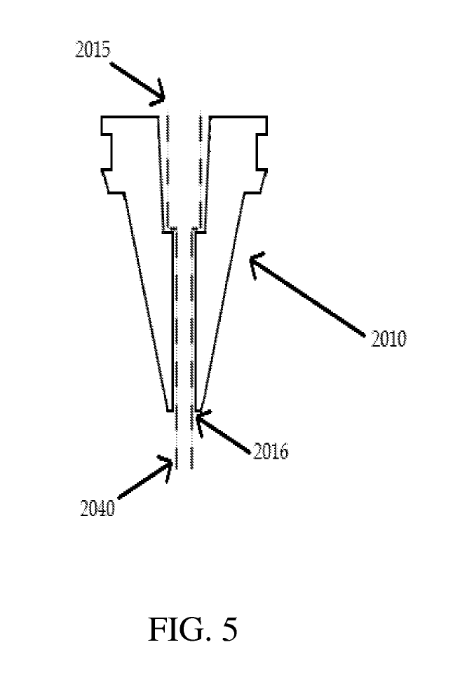

FIG. 5 shows a cut-away side view of the inner body 2010 that comprises the first access port.

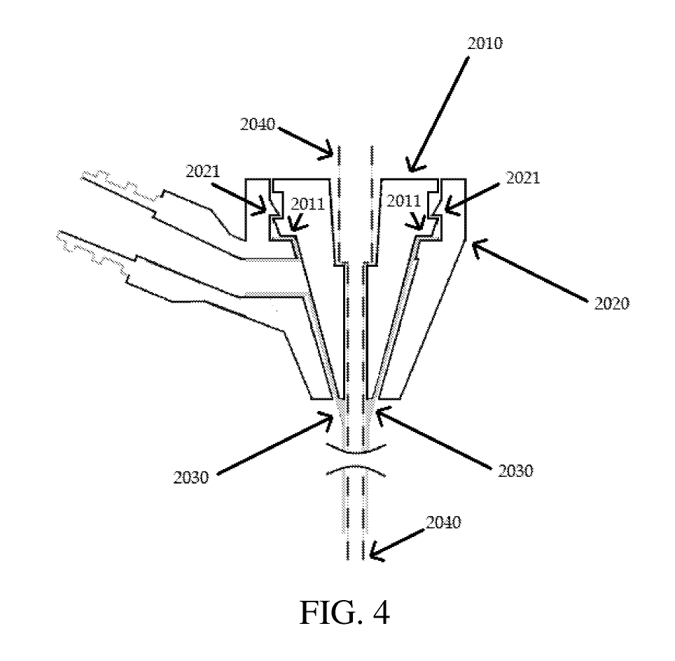

A distal opening 2016 provides an avenue through which a first cannula can pass, said first cannula being comprised by an insertable hypodermic needle or another insertable medical device, represented in this embodiment by the two dashed lines 2040 from FIG. 4.

Once said insertable medical instrument is in place, a proximal opening 2015 is adapted to allow external devices or materials to be directed into the first cannula.

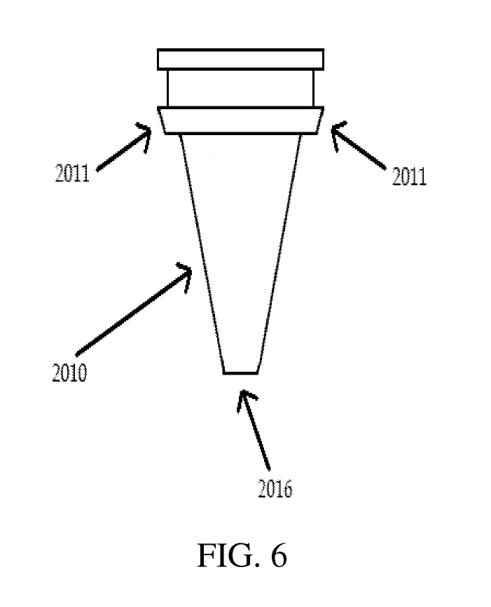

FIG. 6 shows a side view of the inner body 2010 that comprises the first access port. Said inner body comprises a distal opening 2016. Said inner body 2010 comprises an opposite-angle band 2011 which is adapted to secure said inner body 2010 with the outer body 2020 through interaction with the angled pegs 2021 on said outer body 2020 (shown in FIGS. 4, 8, 9, and 10).

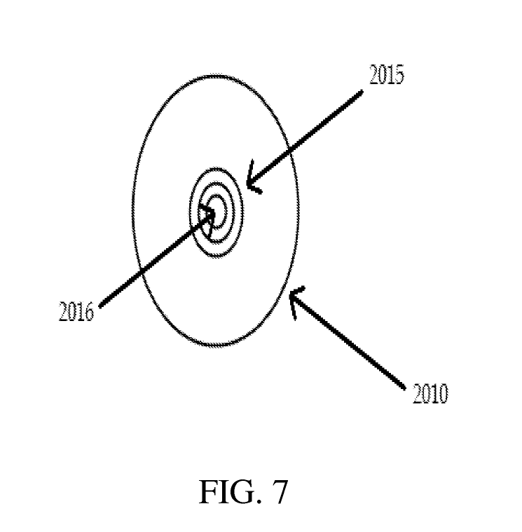

FIG. 7 shows a top view of the inner body 2010 that comprises the first access port. This view shows both the proximal opening 2015 and the distal opening 2016.

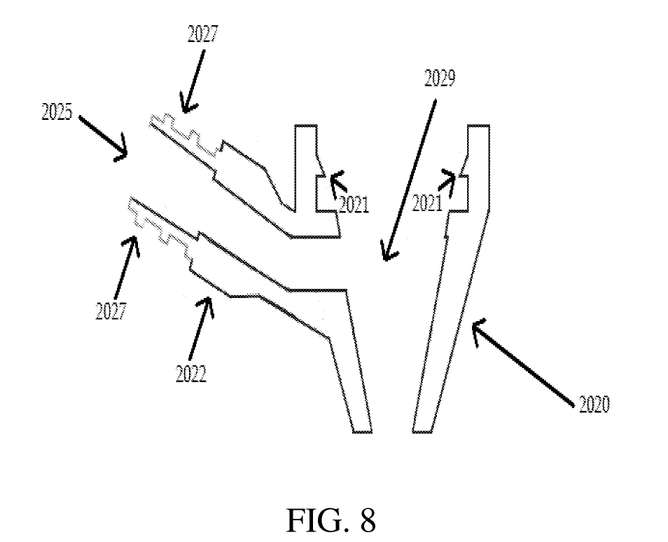

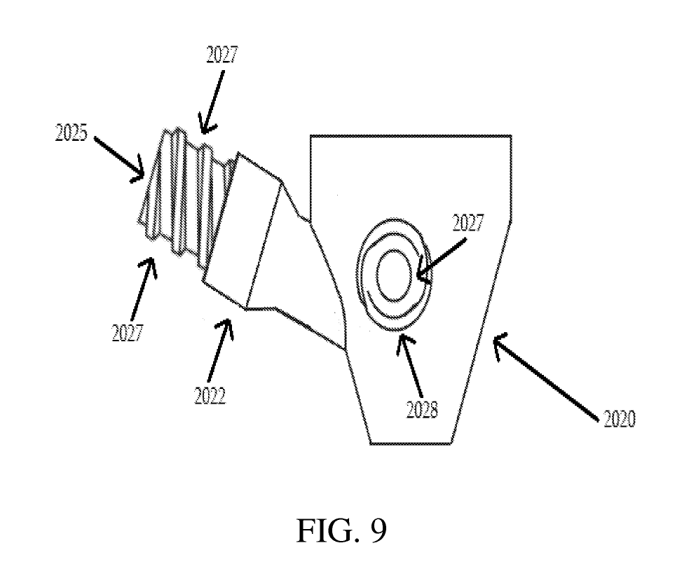

FIG. 8 shows a cut-away side view of the outer body 2020 that comprises the first access port. Said outer body 2020 comprises a proximal end 2022 which also serves as the proximal end of the second access port. Said proximal end 2022 comprises a proximal opening 2025, and is also adapted to be capable of being secured to other objects or devices through the use of a screw shape 2027. The inner body 2010 in FIGS. 4, 5, 6, and 7 is adapted to fit into a space 2029 within said outer body, and to be secured through interaction with the angled pegs 2021.

FIG. 9 shows a side view of the outer body 2020 that comprises the first access port. Said outer body comprises a proximal end 2022 which also serves as the proximal end of the second access port. Said proximal end 2022 is adapted to be capable of being secured to other objects or devices through the use of a screw shape 2027.

Said outer body further comprises a sub-port 2028, said sub-port comprising a screw shape 2027, giving said sub-port the capability of being secured to a filter (not shown) or other device or object.

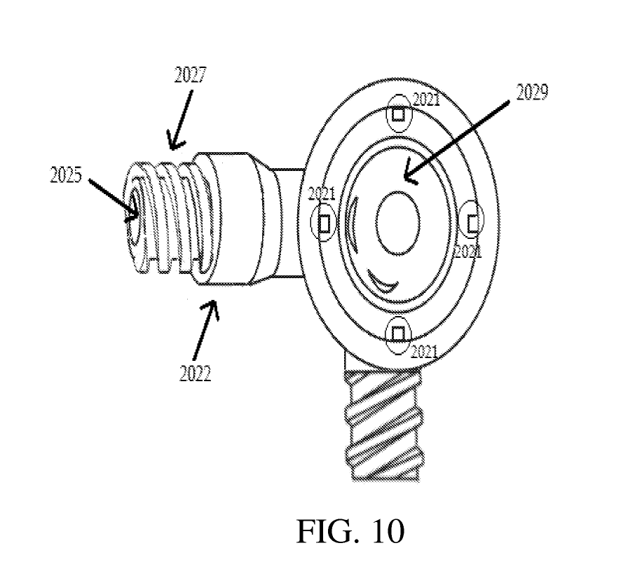

FIG. 10 shows a top view of the outer body 2020 that comprises the first access port. From this view, the angled pegs 2021 within the outer body 2020 are visible, as is the space 2029 into which the inner body 2010 in FIGS. 4, 5, 6, and 7 is adapted to fit.

Said outer body comprises a proximal end 2022 which also serves as the proximal end of the second access port. Said proximal end 2022 is adapted to be capable of being secured to other objects or devices through the use of a screw shape 2027.

Said outer body further comprises a sub-port 2028, said sub-port comprising a screw shape 2029, giving said sub-port the capability of being secured to a filter or other device or object.

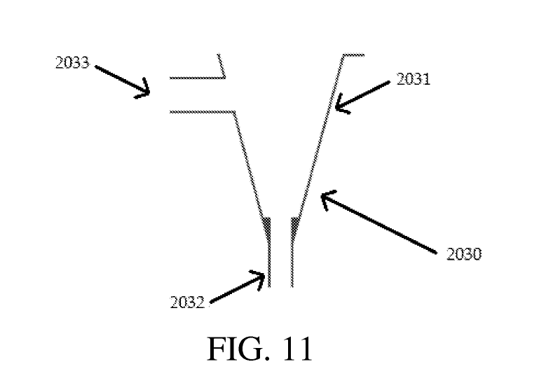

FIG. 11 shows a cut-away side view of the catheter sheath 2030 that comprises the second cannula.

Said catheter sheath 2030 comprises an elastic membrane, said membrane comprising a top part 2031 which is adapted to surround the inner body 2010 (shown in FIGS. 4, 5, 6, and 7) and a bottom part 2032 which is adapted to surround the first cannula. Said membrane is adapted to change shape in response to internal pressure, such as pressure created by viscous materials or other substantive materials which are directed in between said membrane and said inner cannula.

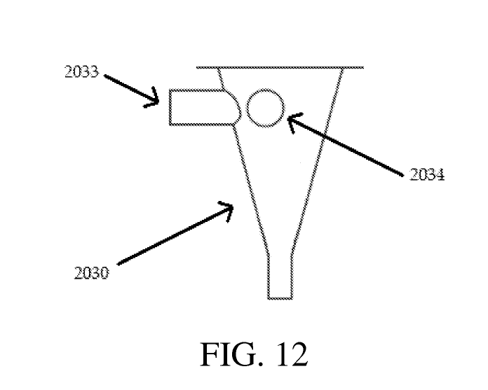

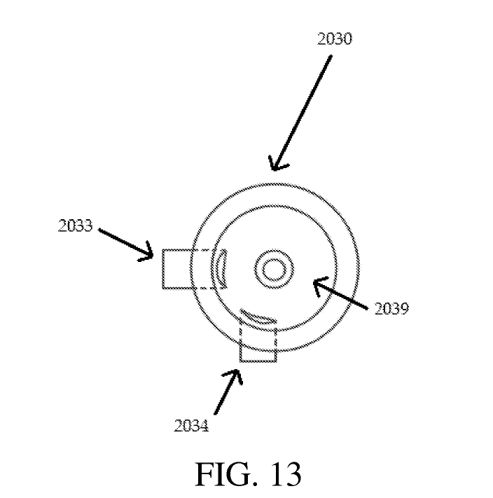

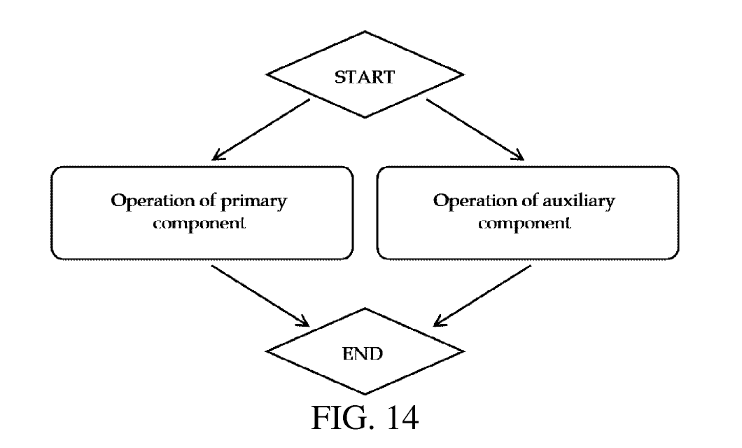

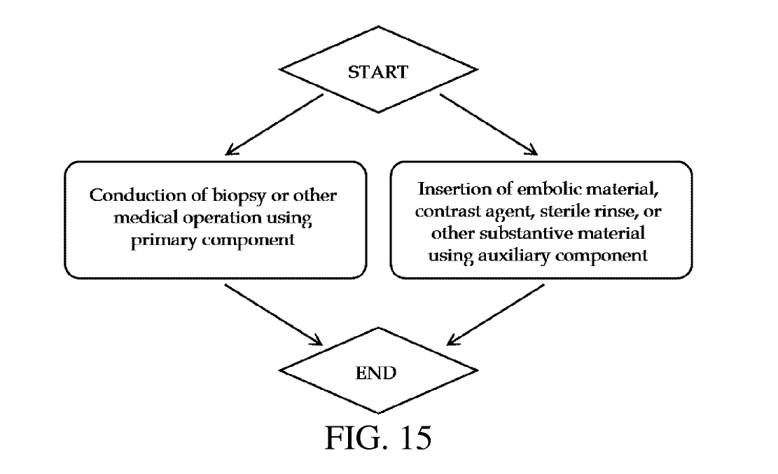

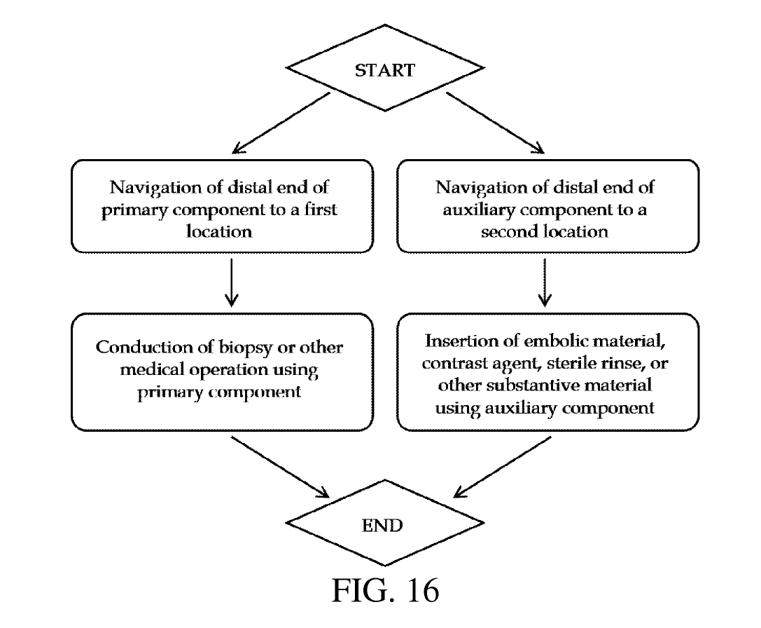

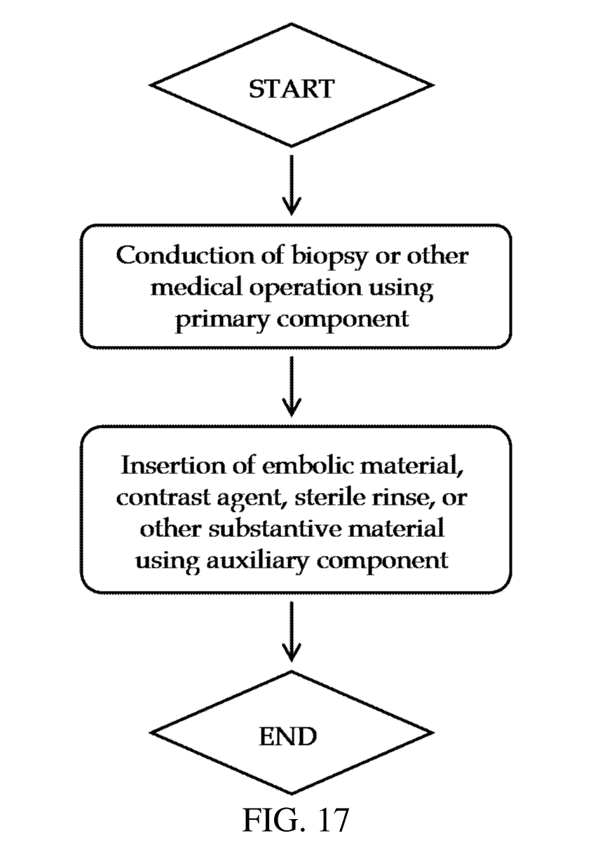

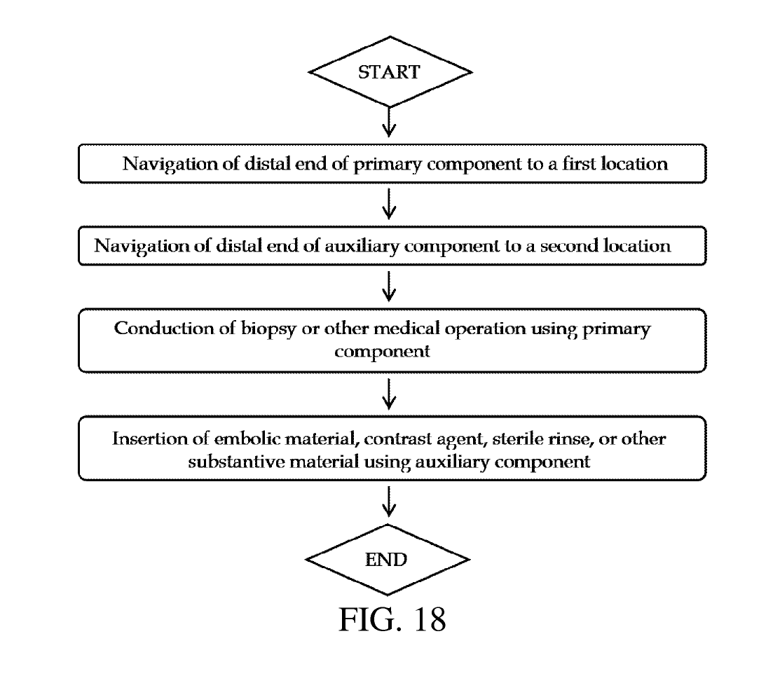

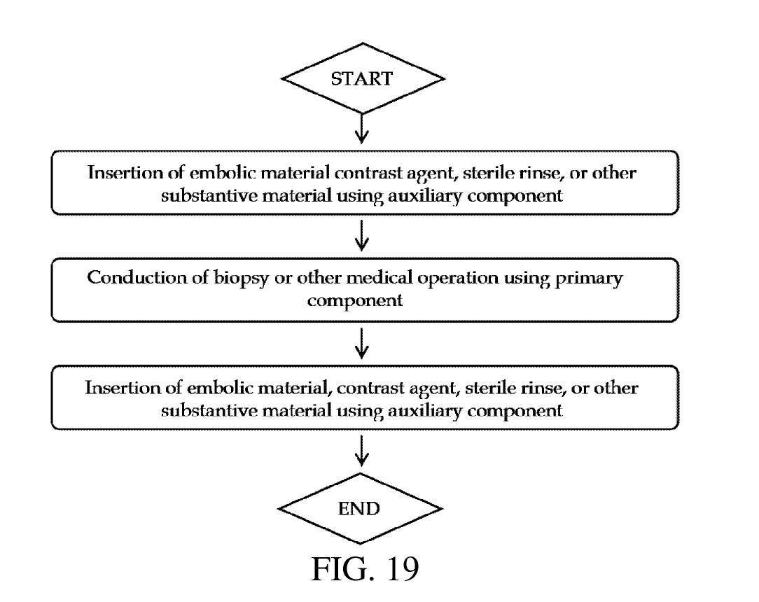

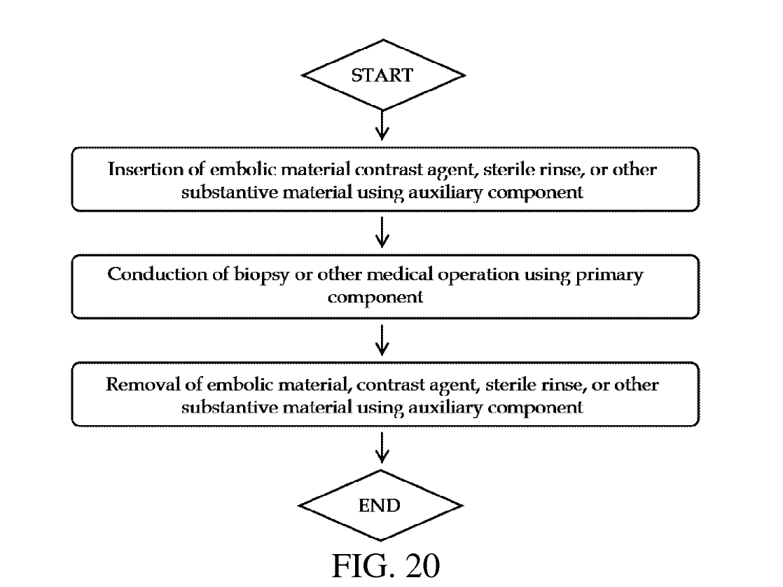

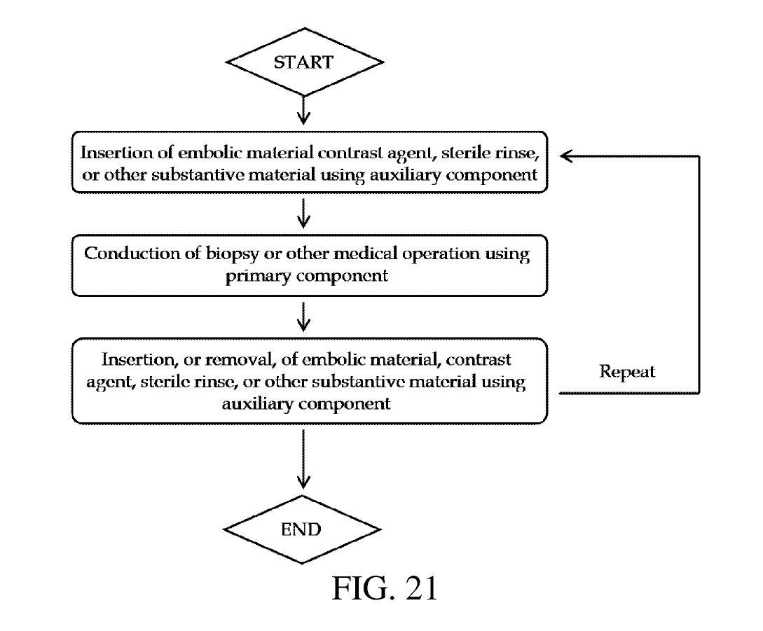

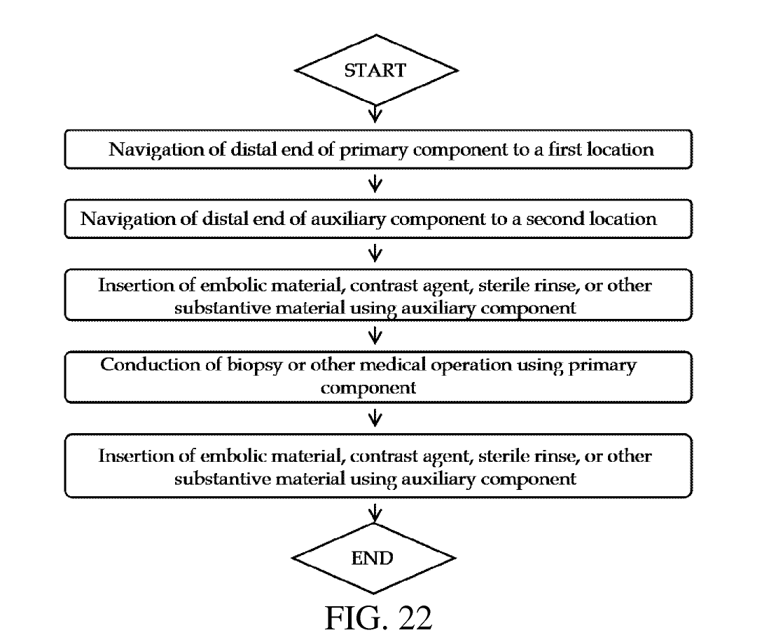

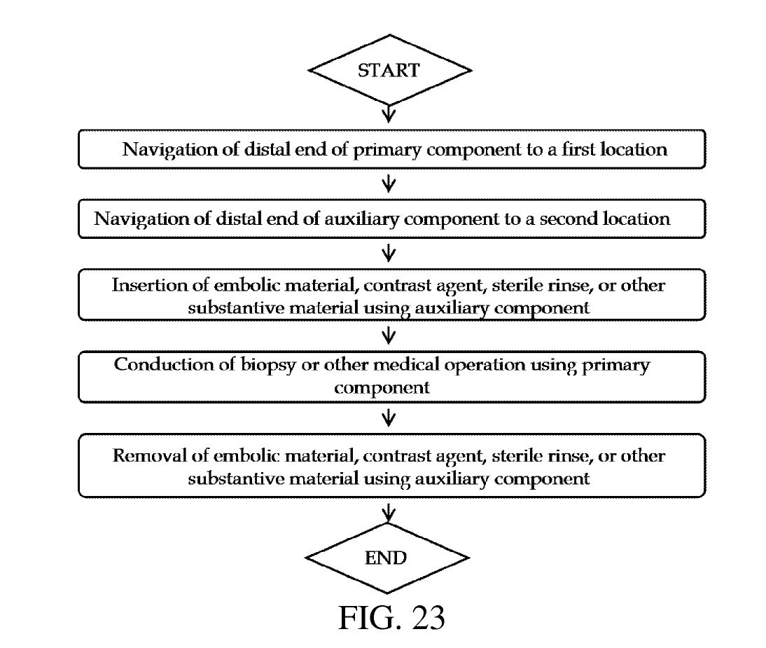

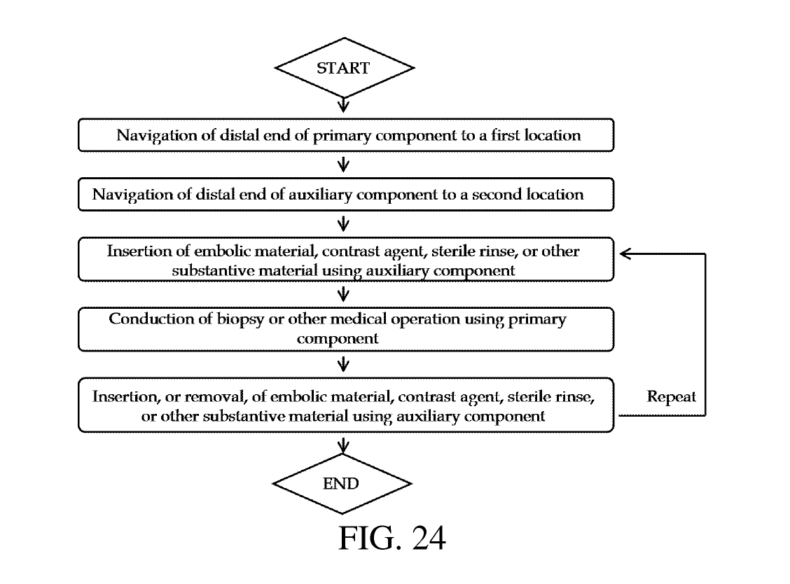

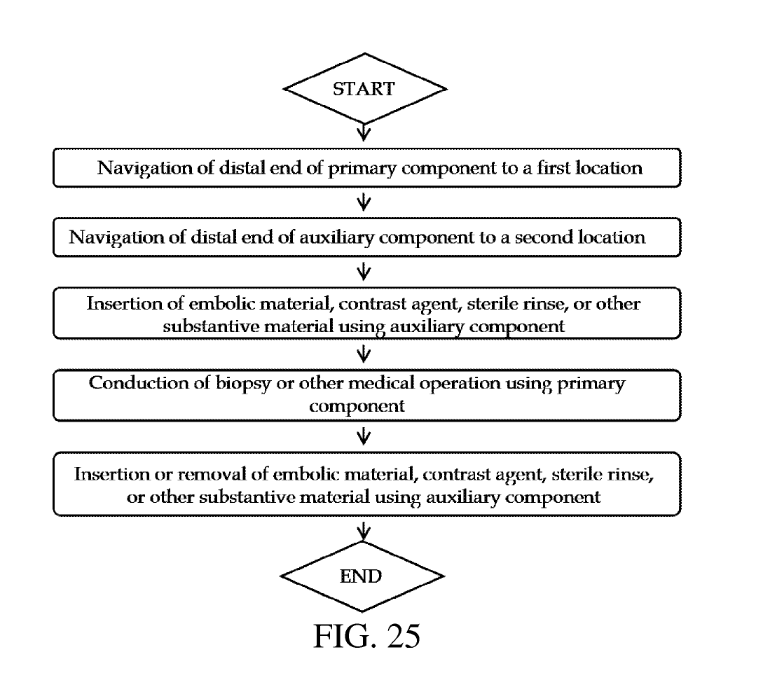

Said catheter sheath also comprises adaptations which enable it to better fit within the space between the inner body and the outer body, such adaptations comprising a port opening 2031 and a sub-port opening (shown in FIGS. 12 and 13); said catheter sheath also is adapted to extend out of the distal opening 2026 of the outer body 2020 (shown in FIGS. 4, 8, 9, and 10).

FIG. 12 shows a side view of the catheter sheath 2030 that comprises the second cannula.

Said catheter sheath comprises additional adaptations which enable it to better fit into the space the inner body and the outer body. Said adaptations comprise a port opening 2033 which is flush with the inner surface of the proximal opening 2025 of the outer body 2020 (shown in FIGS. 4, 8, 9, and 10), and a secondary port opening 2034 which is flush with the inner surface of the sub-port 2028 of the outer body 2020 (shown in FIGS. 4, 8, 9, and 10).

FIG. 13 shows a top view of the catheter sheath 2030 that comprises the second cannula. A space 2039 in the catheter sheath is adapted to fit the inner body 2010 within it.

Said catheter sheath comprises additional adaptations which enable it to better fit into the space the inner body and the outer body, said adaptations comprising a port opening 2033 and a sub-port opening 2034.

FIG. 14 shows a representative method of utilizing the system, said steps comprising the concurrent operating of a primary component along with the operating of an auxiliary component.

FIG. 15 shows a representative method of utilizing the system, said steps comprising the concurrent conducting of a biopsy or other medical operation using a primary component along with the inserting of embolic material, contrast agent, sterile rinse, or other substantive material using an auxiliary component.

FIG. 16 shows a representative method of utilizing the system, said steps comprising the concurrent navigating of a distal end of a primary component to a first location along with the navigating of a distal end of an auxiliary component to a second location, followed by the concurrent conducting of a biopsy or other medical operation using the primary component along with the inserting of embolic material, contrast agent, sterile rinse, or other substantive material using the auxiliary component.

FIG. 17 shows a representative method of utilizing the system using consecutive steps, said steps comprising the operating of a primary component and the operating of an auxiliary component.

FIG. 18 shows a representative method of utilizing the system using consecutive steps, said steps comprising the navigating of a distal end of a primary component to a first location, the navigating of a distal end of an auxiliary component to a second location, the conducting of a biopsy or other medical operation using the primary component, and the inserting of embolic material, contrast agent, sterile rinse, or other substantive material using the auxiliary component.

FIG. 19 shows a representative method of utilizing the system using consecutive steps, said steps comprising the inserting of embolic material, contrast agent, sterile rinse, or other substantive material using the auxiliary component, the conducting of a biopsy or other medical operation using the primary component, and the inserting of embolic material, contrast agent, sterile rinse, or other substantive material using the auxiliary component.

FIG. 20 shows a representative method of utilizing the system using consecutive steps, said steps comprising the inserting of embolic material, contrast agent, sterile rinse, or other substantive material using the auxiliary component, the conducting of a biopsy or other medical operation using the primary component, and the removing of embolic material, contrast agent, sterile rinse, or other substantive material using the auxiliary component.

FIG. 21 shows a representative method of utilizing the system using consecutive steps, some or all of which are capable of being repeated in sequence more than once, said steps comprising the inserting of embolic material, contrast agent, sterile rinse, or other substantive material using the auxiliary component, the conducting of a biopsy or other medical operation using the primary component, and the inserting or removing of embolic material, contrast agent, sterile rinse, or other substantive material using the auxiliary component.

FIG. 22 shows a representative method of utilizing the system using consecutive steps, said steps comprising the navigating of a distal end of a primary component to a first location, the navigating of a distal end of an auxiliary component to a second location, the inserting of embolic material, contrast agent, sterile rinse, or other substantive material using the auxiliary component, the conducting of a biopsy or other medical operation using the primary component, and the inserting of embolic material, contrast agent, sterile rinse, or other substantive material using the auxiliary component.

FIG. 23 shows a representative method of utilizing the system using consecutive steps, said steps comprising the navigating of a distal end of a primary component to a first location, the navigating of a distal end of an auxiliary component to a second location, the inserting of embolic material, contrast agent, sterile rinse, or other substantive material using the auxiliary component, the conducting of a biopsy or other medical operation using the primary component, and the removing of embolic material, contrast agent, sterile rinse, or other substantive material using the auxiliary component.

FIG. 24 shows a representative method of utilizing the system using consecutive steps, some or all of which are capable of being repeated in sequence more than once, said steps comprising the navigating of a distal end of a primary component to a first location, the navigating of a distal end of an auxiliary component to a second location, the inserting of embolic material, contrast agent, sterile rinse, or other substantive material using the auxiliary component, the conducting of a biopsy or other medical operation using the primary component, and the inserting or removing of embolic material, contrast agent, sterile rinse, or other substantive material using the auxiliary component.

FIG. 25 shows a representative method of utilizing the system using consecutive steps, said steps comprising the navigating of a distal end of a primary component to a first location through a cannula-style barrier, the navigating of a distal end of an auxiliary component to a second location, the inserting of embolic material, contrast agent, sterile rinse, or other substantive material using the auxiliary component, the conducting of a biopsy or other medical operation using the primary component, the withdrawing of a distal end of a primary component from said first location through a cannula-style barrier, and the inserting or removing of embolic material, contrast agent, sterile rinse, or other substantive material using the auxiliary component.

I claim:

1. A system comprising a primary component, said primary component comprising an instrument or path, and an auxiliary component, said auxiliary component comprising an instrument or path, which are adapted to work in concert and which are adapted to perform primary and auxiliary tasks upon physical locations which are in close proximity to one another, said primary component comprising a distal end and a proximal end, said auxiliary component comprising a distal end and a proximal end, said distal end of said primary component and said distal end of said auxiliary component being adapted to be capable of being positioned in close proximity to one another.

2. The system as in claim 1, wherein said instrument or path comprises a hypodermic needle, a biopsy apparatus, or a fluid delivery tube.

3. The system as in claim 1, wherein:
said primary component comprises a first access port and a first cannula which interface with one another, and
said auxiliary component comprises a second access port and a second cannula which interface with one another.

4. The system as in claim 3, wherein:
said first cannula comprises a proximal end and a distal end,
said second cannula comprises a proximal end and a distal end,
said distal ends of each cannula are adapted to be capable of being positioned in close proximity to one another,
said proximal end of the first cannula is adapted to interface with the distal end of the first access port, and
said proximal end of the second cannula is adapted to interface with the distal end of the second access port.

5. The system as in claim 4, wherein the first access port is adapted to receive a medical instrument, said medical instrument comprising the first cannula.

6. The system as in claim 5, wherein said medical instrument comprises a hypodermic needle or biopsy apparatus.

7. The system as in claim 6, wherein said medical instrument is adapted to accommodate a second medical instrument.

8. The system as in claim 7, wherein said second medical instrument comprises a hypodermic needle or biopsy apparatus.

9. The system as in claim 4, wherein said first cannula and said second cannula are integrated into a combined cannula needle.

10. The system as in claim 4, wherein said first cannula comprises an inner cannula and said second cannula comprises an outer cannula.

11. The system as in claim 4, wherein said outer cannula will be adapted to change shape in response to internal pressure, such as pressure created by viscous materials or other substantive materials which are capable of being directed in between said membrane and said inner cannula.

12. A method of utilizing the system, comprising a step of operating the primary component and a step of operating the auxiliary component, either concurrently or consecutively.

13. A method as in claim 12, wherein the step of the primary component comprises any method of conducting a biopsy or other medical operation.

14. A method as in claim 12, wherein the step of operating the auxiliary component comprises any method of inserting a type of substantive material.

15. A method as in claim 12, wherein operating the primary component comprises the step of navigating the distal end of said primary component to a first location, and operating the auxiliary component comprises the step of navigating the distal end of said auxiliary component to a second location which is in close proximity to said first location.

16. A method as in claim 12, comprising an additional step of utilizing the auxiliary component to insert substantive material, and the additional step of utilizing the primary component to conduct a biopsy or other medical operation.

17. A method as in claim 16, comprising an additional step of utilizing the auxiliary component to deliver substantive material to the area in which said biopsy or other medical operation was conducted.

18. A method as in claim 16, comprising an additional step of utilizing the auxiliary component to remove substantive material.

19. A method as in claim 12, wherein the method of navigating all or part of said primary component to a first location comprises a step of navigating said primary component through a cannula-style barrier.

20. A method as in claim 19, wherein the method comprises an additional step of withdrawing said primary component through said cannula-style barrier.
